(19) United States
(12) Patent Application Publication
(10) Pub. No.: US 2016/0264998 A1
(43) Pub. Date: Sep. 15, 2016

(54) METHOD FOR TRANSFORMATION OF PROKARYOTE OR EUKARYOTE USING AMINOCLAY

(71) Applicant: INDUSTRY FOUNDATION OF CHONNAM NATIONAL UNIVERSITY, Gwangju (KR)

(72) Inventors: Geun Joong KIM, Gwangju (KR); Hyoung An CHOI, Gwangju (KR); Dea Eun CHEONG, Gwangju (KR); Young Chul LEE, Gyeongsangnam-do (KR); Hyun Jae SHIN, Jeollanam-do (KR)

(21) Appl. No.: 14/410,062

(22) PCT Filed: Jul. 17, 2014
(86) PCT No.: PCT/KR2014/006472

§ 371 Date: Dec. 20, 2014

(30) Foreign Application Priority Data
Jul. 26, 2013 (KR) 10-2013-0088728

(51) Int. Cl. C12N 15/87

(52) U.S. Cl. CPC C12N 15/87

(57) ABSTRACT

The present invention relates to a method for transformation of a prokaryote or an eukaryote using aminoclay wherein the method includes mixing aminoclay, foreign nucleic acids, and a prokaryote or an eukaryote, and a composition and a kit for transformation of a prokaryote or an eukaryote containing aminoclay, in which the method for transformation of a prokaryote or an eukaryote using aminoclay according to the present invention has a simple and efficient process not requiring expensive equipments, and thus the method for transformation of a prokaryote or an eukaryote according to the present invention can be usefully employed for development of useful microbial strains that can be used in an industry of renewable energy, a food industry, and also for producing high added-value biomaterials and cosmetic raw materials.

### TECHNICAL FIELD

The present invention relates to a method for transformation of a prokaryote or an eukaryote using aminoclay, specifically, a method for transformation of a prokaryote or an eukaryote including mixing aminoclay, foreign nucleic acids, and a prokaryote or an eukaryote, and a composition and a kit for transformation of a prokaryote or an eukaryote containing aminoclay.

### BACKGROUND ART

Cell transformation with a foreign DNA is an essential step of genetic modification of microorganisms such as Gram-negative and Gram-positive bacteria, yeast, or fungus. Examples of the most widely used transformation method include a heat shock treatment, a chemical treatment (e.g., a treatment with divalent metal ions, a membrane-permeabilization agent, or antibiotics), a mechanical treatment (e.g., milling and ultrasonication), electroporation, and a virus-/nanocarrier-mediated approach. Typical treatment methods exhibit transformation efficiency which is above a certain level depending on properties of cell membrane, cell wall, or cell physiology, stress induced by pre-treatment, death ratio, or the like. However, a technique which can be generally applied regardless of a classification and physiology of cells does not exist. Among the techniques, physical incorporation into a cell (using frictional force or shear stress) involving winding or adsorption of DNA on nanofibers by using nanofibers or whiskers of chrysotile, carbon nanotube, silicon carbide, sepiolite, or the like have been broadly studied in recent years as a fast and cost-effective method for transferring DNA to a cell, in particular, bacteria. Further, such method using nano substances has a technical characteristic that it can be generally applied to a broad range of host. However, in spite of those advantages, as a DNA carrier, the needle type material for nano infiltration induces cancer or is believed or known as a inflammation inducing agent, and therefore it may lead to unexpected stress in bacteria (e.g., causing self defense system or mutation, or even death in an extreme case). Due to such physicochemical risk, it is preferred to have nanomaterials with a non-infiltrating or non-invasive property for application in a living organism (body). As such, for further industrial application in industrial or medical use, an alternative method for microorganism transformation which is free of any risk and has a biocompatibility or safety is highly required.

Aminoclay (metal phyllosilicate functionalized with 3-aminopropyl) was developed in 1997. It is synthesized by dropwise addition, i.e., one-pot sol-gel reaction, of 3-aminopropyl triethoxysilane (APTES) to an ethanol solution containing cationic metals (Mg²⁺ or Ca²⁻, in general) at atmospheric conditions (room temperature and normal pressure). The consequently obtained product is referred to as magnesium- and calcium-aminoclay, respectively. The aminoclay consists of metal cations in unit structure backbone, which are inserted between covalently-linked functional groups functionalized with an amino group as induced by hydrolysis and condensation of APTES. White aminoclay with pKa of 9.6 which has been produced accordingly is easily dissociated in an aqueous solution, and after an ultrasonic pre-treatment for 5 minutes, it becomes transparent and water soluble. Novel bioinorganic nanostructures are produced such that they can have enhanced thermal and mechanical stability while still maintaining intrinsic characteristics. Driving force for inducing a self-assembled (nano)subject is mainly related to zeta potential charged with positive charges of de-aminated aminoclay nanoparticles, and it is based on by repulsive force of protonated amine groups in an aqueous solution. The organic-building block of amino clay exhibits average fluid dynamic diameter of about 30 nm. When an aminoclay-substrate is wrapped or coated with a target anionic material, condensed precipitates are yielded, representing that, in an equilibrium state, the target material is densely and evenly covered by an organic-building block of aminoclay in aqueous solution. Thus, the organic-building block of aminoclay has a strong interaction with a molecular structure which is negatively charged in an aqueous solution (i.e., biomaterial like DNA and RNA). As a result, DNA wrapped with extremely thin aminoclay coating can be provided. Although it has been expected to be used as a delivery vehicle for non-viral gene transfection, similar to an enzyme/lipid complex, or intermediate plate structure of bacteriorhodopsin, the method has not been actually applied to transformation of microorganism. The structural characteristic based on above properties suggests a new possibility that DNA in a nano construct coated with aminoclay is protected from an attack by endonuclease. This also suggests a new possibility that the disadvantages remained as a problem in transformation method using typical nano structure can be basically overcome. Further, the aforementioned water soluble aminoclay in appropriate concentration range does not exhibit any toxicity to mammalian cells and exhibits very minor ecotoxicity to Pseudokirchneriella subcapitata and Daphnia magna. Those results present a possibility that, in an appropriate concentration range, aminoclay or a molecule adsorbed onto aminoclay can be delivered to a cell membrane with slightly-induced physical damages but without inducing cell death. For such process, it can be considered to introduce frictional force for having more effective delivery or internalization (i.e., applying physical force by mixing aminoclay composite and cells). On the contrary, it is also possible that toxic marine algae are selectively controlled by adjusting the concentration to desired level.

According to previous studies, it has been reported that the interaction between an aminoclay organic-building block and bacteria plays an important role of de-stabilizing cell membrane or cell wall due to electrostatic interaction. Specifically, because the aminoclay nanoparticles induce rapid adsorption on cell surface of bacteria and subsequent penetration into intracellular regions, damage (cracks) in a cell membrane is caused. Accordingly, it means that the nanoparticles can function as an anti-bacterial agent when they are present at or above the appropriate concentration. If a loading amount of aminoclay can be maintained at an appropriate level for transformation without having serious damages, based on such unique properties of penetrating cell membrane by using positively charged structure and the possibility of protecting DNA from enzymatic restriction within a hybrid nanomaterial obtained by a self-assembled structure, the aminoclay may be employed as an agent for bacteria transformation. If necessary, it is possible to introduce frictional force to this process for enhancement of efficiency.

In Korean Patent Registration No. 87-000510, a method of producing transformed microorganisms is disclosed. However, there is no disclosure regarding the method of transforming a prokaryote or an eukaryote using aminoclay as described in the present invention.

### DETAILED DESCRIPTION OF THE INVENTION

### Technical Problems to be Solved

The present invention is devised under the circumstances described above, and the inventors of the present invention confirmed that transfection of Gram-negative E. coli and Gram-negative Streptococcus mutans, yeast, and fungus can be successfully achieved by using aminoclay, and they consequently established a method for transforming a microorganism which is simple and has high reproducibility and excellent survival ratio after transformation. The present invention is completed accordingly.

### Technical Means for Solving the Problems

In order to solve the aforementioned problem, provided by the present invention is a method for transformation of a prokaryote or an eukaryote using aminoclay wherein the method includes mixing aminoclay, foreign nucleic acids and a prokaryote or an eukaryote.

Also provided by the present invention is a composition for transformation of a prokaryote or an eukaryote containing aminoclay.

Also provided by the present invention is a kit for transformation of a prokaryote or an eukaryote containing aminoclay.

### Advantageous Effect of the Invention

The method for transformation of a prokaryote or an eukaryote using aminoclay according to the present invention has a simple and efficient process. Therefore, the method for transforming bacteria, fungi, or yeast according to the present invention can be usefully employed for development of useful microbial strains which can be used in an industry of renewable energy, a food industry, a pharmaceutical industry, and also for producing high added-value materials and cosmetic raw materials.

### BRIEF DESCRIPTION OF THE DRAWINGS

FIG. 1 is a drawing illustrating the process of bacterial transformation. (A) Method for adsorbing DNA on inorganic fibers (i.e., Yoshida effect transformation). (B) Method for constructing a hybrid with the aforementioned structure by wrapping DNA with aminoclay organic-building block.

FIG. 2 is a drawing illustrating the transformation efficiency plotted against the amount of plasmid DNA or spreading time. (A) Effect of plasmid DNA concentration on transformation. (B) Influence of spreading time on transformation. Bar graph indicates an average±SD of three independent tests.

FIG. 3 is a drawing illustrating a fluorescent microscopic image of transformed cells in a liquid medium. (A) Green fluorescence image of E. coli XL1-Blue having pBBR122-gfp. (B) Green fluorescence image of Streptococcus mutans having pBBR122-gfp. Both cells were transformed by spreading with a mixture of calcium-aminoclay/plasmid. The images were captured with a DP71 digital camera using DIC and 500 ms exposure for fluorescent image, and adjusted with a software (DP-BSW, TOMORO digital image package).

### BEST MODE(S) FOR CARRYING OUT THE INVENTION

In order to achieve the purpose of the present invention, the present invention provides a method for transformation of a prokaryote or an eukaryote which includes mixing aminoclay, foreign nucleic acids and a prokaryote or an eukaryote.

The term “aminoclay” indicates a clay-like material, and it is one type of organoclay. Specifically, it is a material produced by a sol-gel reaction between metal cations (Mg²⁺, Ca²⁺, Zn²⁺, Mn²⁺, Al³⁺ and Fe³⁺) and (3-aminopropyl)triethoxysilane [APTES], which has a structure in which metal cations are present at center and are surrounded by a 2:1 trioctahedral structure or form a pair with 1:1 octahedral structure with covalently-linked amine groups. In the present invention, the aminoclay may consist of the chemical component [H₂N(CH₂)₃]₈Mg₆Si₈O₁₆(OH)₄. Such amino clay allows higher efficiency for physical introduction of foreign nucleic acids to a prokaryote or an eukaryote and higher survival ratio after the transformation. As for the aminoclay, either commercially available products or synthesized products can be used.

For the transformation method according to one embodiment of the present invention, the aminoclay was produced as water soluble aminoclay as a result of mixing magnesium chloride hexahydrate (MgCl₂.6H₂O) and (3-aminopropyl)triethoxysilane (APTES).

In the present invention, the term “foreign nucleic acids” collectively indicates non-endogenous DNA or RNA molecules. Specifically, it may indicate a compound including nucleotides as a basic constitutional unit of a nucleic acid molecule and it may indicate a nucleic acid molecule which has been engineered to modify a genotype, and thus a related phenotype of a living organism, in particular. According to the purpose of the present invention, the foreign nucleic acids can be nucleic acids that are capable of modifying a phenotype or a property of a prokaryote or an eukaryote to which the foreign nucleic acids are incorporated. Preferably, it can be the nucleic acids that are capable of enhancing usefulness (e.g., over-production of a pharmaceutical protein or a high added-value compound based on metabolic engineering and/or genetic circuit re-designing) of a target prokaryote or eukaryote, promoting the production, or enhancing the metabolic capacity in an appropriate environment.

Preferred examples of the foreign nucleic acids of the present invention include, although not particularly limited thereto, a vector to which a promoter, ribosome binding site or a gene for transformation, an antibiotic-resistant gene, or the like are operably linked.

The term “operably linked” means that a regulatory sequence for expression is linked such that it can control the transcription of a polynucleotide sequence which encodes a constitutional element such as nucleic acids for transformation, and it may include maintaining an accurate translation frame for generating a constitutional element such as proteins for transformation which is encoded by a regulatory sequence for expression including a promoter and ribosome binding site. Further, the aforementioned “vector” indicates a nucleic acid construct comprising an essential regulatory element that is operably linked so as to express a target protein in a suitable host cell. Further, the aforementioned “antibiotic-resistant gene” indicates a constitutional element which is expressed in conjunction with nucleic acids for transformation so as to determine and select the clone with recombinant vectors during transformation. Although it is not particularly limited, a person skilled in the art may use a suitable antibiotic-resistant gene depending on the purpose described above.

The term “prokaryote” as described herein indicates an organism having a primitive cellular nucleus called prokaryotic nucleus and contrary to an eukaryote. It is generally composed of a single cell, and examples thereof include a photosynthetic microorganism and archaea. Their DNA is not surrounded by a membrane. In cytoplasm, it is present in molecular state and it is characterized by not having a cell organelle structure like mitochondria.

According to the transformation method of one embodiment of the present invention, the prokaryote can be bacteria, and preferred examples thereof include Streptococcus mutans, E. coli (Escherichia coli), Staphylococcus aureus, Streptococcus pyogenes, Corynebacterium glutamicum, Propionibacteria acnes, Bacillus subtilis, Pseudomonas aeruginosa, Lactobacillus plantarumm, and Agrobacterium tumefaciens, but not limited thereto.

The term “eukaryote” as described herein indicates an organism having eukaryotic cells, and it is a general name of the cells which have various cellular organelles as represented by a nucleus in a cell. Examples thereof include typical multicellular organisms and also various algae including blue algae and green algae, monocellular organisms like yeast, and fungi like mold.

According to the transformation method of one embodiment of the present invention, the eukaryote can be yeast or lower fungus, and preferred examples thereof include Saccharomyces cerevisiae, Schizosaccharomyces pombe, Pichia pastoris, Dictyostelium discodeum, Candida albicans, and Aspergillus oryzae, but not limited thereto.

The aminoclay of the present invention may be in hydrated form. As described herein, the term “hydrated” represents a state in which a solute molecule or an ion molecule is surrounded by a solvent to behave like a single molecule when the solvent is water. Specifically, it represents a state in which particles dispersed in water, solutes, ions, or colloid molecules in an aqueous solution attract a water molecule and exhibit a property as a group.

According to the transformation method of one embodiment of the present invention, the aminoclay is hydrated by mixing it at concentration of 10 mg/ml in distilled water for 24 hours.

According to the transformation method of one embodiment of the present invention, mixing of the hydrate with nucleic acids (i.e., DNA or RNA) can be performed by vortexing, but it is not limited thereto.

The term “vortexing” as described herein means a mixing treatment by which whirlpool is created in a subject solution by vibration from outside or by using a magnetic bar or the like.

The vortexing of the present invention can be performed for 30 to 120 seconds, preferably for 40 to 80 seconds, and more preferably for 60 seconds, but it is not limited thereto.

According to the present invention, culturing the mixture obtained after the aforementioned mixing step with a host for transformation may be further comprised. Culturing the mixture with a host may include spreading a mixture-host solution on a plate medium (to provide frictional force for efficient introduction of nucleic acids to a cell).

The term “plate medium” as described herein means a solid medium which is obtained by solidifying a liquid medium to a plane shape by using agar, gelatin, or the like. Further, the term “spreading” as described herein means one of the methods for inoculating microorganisms to a solid medium, and it indicates a process for smearing inoculant onto a solid medium by streaking or planar spreading. According to this process, frictional force between the mixture and cells is provided. Depending on the purpose, a person skilled in the art may easily select and use agar concentration of a plate medium, spreading strength, and volume of the mixture for spreading.

According to the present invention, selecting a transformed prokaryote or eukaryote may be further comprised after the aforementioned culturing of the mixture. According to the examples of the present invention, the transformed prokaryote or eukaryote which has been cultured in the plate medium was subcultured in a liquid medium containing antibiotics by using continuous subculturing method, and the transformant with resistance to antibiotics was selected.

In order to compare the efficiency of the transformation method using aminoclay according to one embodiment of the present invention to that of a transformation method of a related art, the transformation efficiency was determined for a method using heat shock process (Kindle. et al., J Cell Biol. 1989 109 (6 Pt1): 2589-601) or electroporation (Shimogawara. et al., Genetics. 1998 148: 1821-8), which are the representative transformation method of a related art, and also for a method using a commercially available kit (Table 1). As a result, as shown in Table 1, the transformation efficiency using aminoclay was found to be comparable or better than that of any other transformation methods.

The present invention also provides a composition for transformation of a prokaryote or an eukaryote containing aminoclay. The composition for transformation of a prokaryote or an eukaryote according to the present invention may be a composition which further comprises distilled water, nucleic acids for transformation, saline buffer, or a composition for plate medium or the like that are required for transformation of a prokaryote or an eukaryote using aminoclay, but it is not particularly limited thereto.

The prokaryote or eukaryote is as defined above.

The present invention also provides a kit for transformation of a prokaryote or an eukaryote containing aminoclay. The kit of the present invention may be a kit which further comprises distilled water, nucleic acids for transformation, saline buffer, or a composition for plate medium or the like that are required for transformation of a prokaryote or an eukaryote using aminoclay, but it is not particularly limited thereto.

Herein below, the present invention is explained in view of the examples. However, the following examples are given only to illustrate the present invention and by no means the scope of the present invention is limited to them.

### Materials and Methods

### 1. Chemicals

Magnesium chloride hexahydrate (MgCl₂.6H₂O) and calcium chloride dehydrate (CaCl₂.2H₂O) as a cationic metal were purchased from Junsei Chemical Co., Ltd. (Japan) and (3-aminopropyl)triethoxysilane (APTES, C₉H₂₃NO₃Si) as aminosilane for synthesizing aminoclay was purchased from Sigma-Aldrich Company (USA). Luria-Bertani (LB) and brain heart infusion (BHI) medium were obtained from Becton, Dickinson and Company, USA, respectively. For the steps of preparing DNA for transformation, triple-distilled water (>18 mΩ, DI water) was used for all experiment.

### 2. Plasmid, Bacterial Strains, and Conditions for Growth

pBBR122 (Mostafa et. al., 2002. Appl. Environ. Microbiol. 68, 2619-2623), which is a broad host range vector, was used for transformation of E. coli (Escherichia coli) XL1-Blue and Streptococcus mutans (KCTC 3065). The gene encoding the green fluorescent protein GFPuv was amplified by PCR by using a primer set (forward direction primer: 5′-AAAGTACTATGAGTAAAG-3′ (SEQ ID NO: 1) and reverse direction primer: 5′-AACCGAATTCTTATTTGTAG-3′ (SEQ ID NO: 2)) together with pGFPuv plasmid (Clontech, USA) as a template to obtain GFP expression-recombinant vector. The amplified gene and pBBR122 were digested with ScaI and EcoRI and ligation was performed according to a standard method. The construct obtained as a result was named pBBR122-gfp (5.6 kb), and after the cloning, it was used for GFP expression. All recombinant plasmids which have been used for transformation of each host were isolated by using Mini-Prep kit (Promega, USA), and the nucleotide sequence was analyzed and used for the following experiments.

E. coli and Streptococcus mutans carrying the recombinant plasmid were grown in LB and BHI medium, respectively. In order to have various frictional forces during the step of spreading a mixture of the plasmid-aminoclay/cell mixture (i.e., frictional forces between the plasmid-aminoclay mixture present in a liquid layer on solid surface and host cells), the solid plate was prepared to have a varying concentration (2 to 4%) of agar. The antibiotic Kanamycin used as a selection marker was added to an agar plate at a concentration of 25 μg/ml and used for selecting transformed strains.

### 3. Preparation of Aminoclay Stock Solution

Characteristic information about magnesium- and calcium-amino clay including chemical synthesis of aminoclay, transmission electron microscopic image, dynamic light scattering data, Fourier transformed infrared spectrum, and X-ray diffraction pattern of the aminoclay dispersed in triple-distilled water (deionized water) have been disclosed in documents of previous studies (Han et al., 2011. ACS Appl. Mater. Interfaces 3, 2564-2572; Lee et al., 2013. Sci. Rep. 3, 1292 (1-8); Lee et al., 2011. J. Hazard. Mater. 196, 101-108; Lee et al., 2011. J. Hazard. Mater. 190, 652-658; Lee et al., 2012. Adv. Sci. Lett. 6, 882-887). Each aminoclay in an amount of 10 mg was suspended in triple-distilled water (1 ml) for 24 hours for completely dissolve the clay. The aforementioned stock solution was used for every transformation experiment without having a further treatment.

### 4. Protocols for Transformation

Cultured cells of E. coli XL1-Blue (4×10⁸ CFU) and Streptococcus mutans (6×10⁸ CFU) were centrifuged for 10 minutes at 13,000 rpm and collected at early exponential phase of growth. They were then re-suspended in 500 μl of triple-distilled water or phosphate-buffered saline (PBS, pH 7.0). Purified plasmid pBBR122 (0.05, 0.1, 0.25, 0.5 and 1.0 μg) was added to 50 μl solution of hydrated aminoclay (10 mg/ml) and further adjusted to 500 μl with DI water. The resuspended cells (500 μl) were finally mixed and vortexed for 1 minute with the above solution. They were completely mixed and subjected to the reaction (i.e., induced for adsorption on cell surface). The mixture prepared according to the steps described above was spread on an agar plate which comprises LB or BHI solid medium containing 2% agar in which Kanamycin (25 μg/ml) is included. The plate used was dried in advance for 10 minutes on a clean bench. The mixture (100 μl) comprising the cells and aminoclay/plasmid was spread on an agar plate (diameter; 9 cm) for transforming the bacteria with a plasmid. The spreading was performed using a glass spreader until the liquid mixture is completely absorbed into the agar plate. For searching an optimum condition therefor, each sample (3 for each) was prepared and further spread for 30 seconds, 60 seconds, or 120 seconds (under expectation that the transformation frequency is improved by frictional forces). The resultant plate was incubated for 24 hours at 37° C., followed by colony observation. From the well isolated colony, the plasmid was purified by Mini-Prep kit to determine whether or not the transformation has been successfully made. Each experiment was repeated three times and the transformation efficiency was calculated. The average value and standard deviation were calculated by using Sigma Plot Version 10.0 (Systat Software Inc. Chicago, USA).

### 5. Plasmid Stability and Effect on Cell Growth

In order to confirm the effect of treating with aminoclay/plasmid on the plasmid stability and cell growth, two microbial strains transformed with plasmid pBBR122-gfp were cultured in 250 ml flask at 37° C. by using 50 ml of LB medium or BHI liquid medium. The culture was grown for about 80 generations, and while transferring it continuously to another flask with the same conditions, stability of the plasmid and level of cell growth were compared to those of cells transformed by typical method (heat shock) and the control strains without plasmid. To do so, a constant amount of the sample was collected from each culture, and the culture broth diluted to a suitable concentration was spread onto the two same solid media that are different only in terms of the presence of the selection marker Kanamycin (25 μg/ml), i.e., Kanamycin is either present or absent. The plasmid stability during the above step (i.e., loss rate) was determined based on a ratio of cells which exhibit resistance to the antibiotics compared to the entire clones. Final confirmation of the presence or absence of the plasmid was made according to the process of isolating the plasmid DNA by using Mini-Prep kit from the cells which have been cultured in the presence of antibiotics. During the test, the growth delay of the aminoclay-mediated transformed E. coli cells (i.e., inhibited growth caused by nano materials or frictional forces) or possibility of having induced expression of a heat shock protein by stress was compared by using cells which have been transformed with the same plasmid according to a typical transformation method (i.e., heat shock)

### EXAMPLE 1

### Interaction Between Plasmid DNA and Aminoclay

To determine the presence or absence and degree of forming an aminoclay-DNA complex (i.e., DNA wrapped with aminoclay) that is advantageous for transformation, the plasmid with various concentration but having the same aminoclay concentration was incubated for 24 hours at room temperature and the amount of the plasmid DNA wrapped with each aminoclay was examined As it has been generally known, due to positively charged aminoclay surface in a broad pH range (i.e., pH of from 2 to 12) (Chaturbedy et al., 2010. ACS Nano 4, 5921-5929; Lee et al., 2011. J. Hazard. Mater. 196, 101-108), it is expected that there is an easy interaction with the plasmid DNA having negative charge to yield the plasmid DNA coated with aminoclay. As a result of electrophoresis, it was found that the plasmid in an amount of about 2.75 to 3.25 μg/mg or 1.75 to 2.25 μg/mg can be adsorbed to magnesium- and calcium-aminoclay, respectively (i.e., complex is formed). This is a result obtained by utilizing the characteristic of a DNA in complex form, i.e., it does not allow fluorescent staining with EtBr on a gel. This result was determined again by a quantitative analysis of un-bound DNA based on measurement of optical density at 280 nm by using a spectrophotometer. As a result, from the mixture solution of magnesium- and calcium-aminoclay within the concentration range having complete adsorption on the aminoclay (i.e., 2.75 to 3.25 μg plasmid/mg magnesium-aminoclay; 1.75 to 2.25 μg plasmid/mg calcium-aminoclay), the DNA-specific spectrum (260 to 280 nm) was not observed. This is a result demonstrating that, as the DNA in a self-assembled hybrid structure exhibits a stable structure described above and the aminoclay as a coating material serves as armor for the plasmid DNA biomolecule. Further, this structure was protective against the DNA restriction using HindIll restriction enzyme or DNaseI for 30 minutes to 60 minutes, and no DNA fragment was observed (data not shown). Thus, when introduced to a living cells, it is expected to exhibit relatively high transformation efficiency as being protected from restriction enzymes or enzymes for hydrolyzing nucleic acids.

### EXAMPLE 2

### Effectiveness of Amount of Plasmid DNA and Spreading Time for Transformation Efficiency

pBBR122 plasmid widely used as a common transformation vector for various microorganism hosts was arbitrarily selected and transformation capability of the aminoclay for typical Gram-negative (E. coli) and Gram-positive (Streptococcus mutans) cells was evaluated. This is based on the finding by the inventors of the present invention that the aforementioned vector can successfully function in a broad range of Gram-negative host and Streptococcus mutans can also be transformed with the aforementioned vector. According to the result of a preliminary experiment, it was found that the aminoclay/plasmid complex can transform host cells that are harvested at different cell growth phase (i.e., lag phase, exponential growth phase, and cell growth stationary phase). Thus, it was found that the cells at any phase of growth can be used as a competent cell (i.e., host for transformation). However, considering cell physiology and relatively high frequency of transformant, cells at the early exponential growth phase (i.e., optical density at 600 nm is 0.8 to 1.5) were used as competent cells. The competent cells used in the present invention were prepared only by harvesting using centrifuge without adding any additives. On the other hand, a typical transformation method (e.g., heat shock or electroporation) requires complex physical and chemical treatments (i.e., after repeating washing with glycerol solution or metal ion solution, centrifuge is performed under cold conditions).

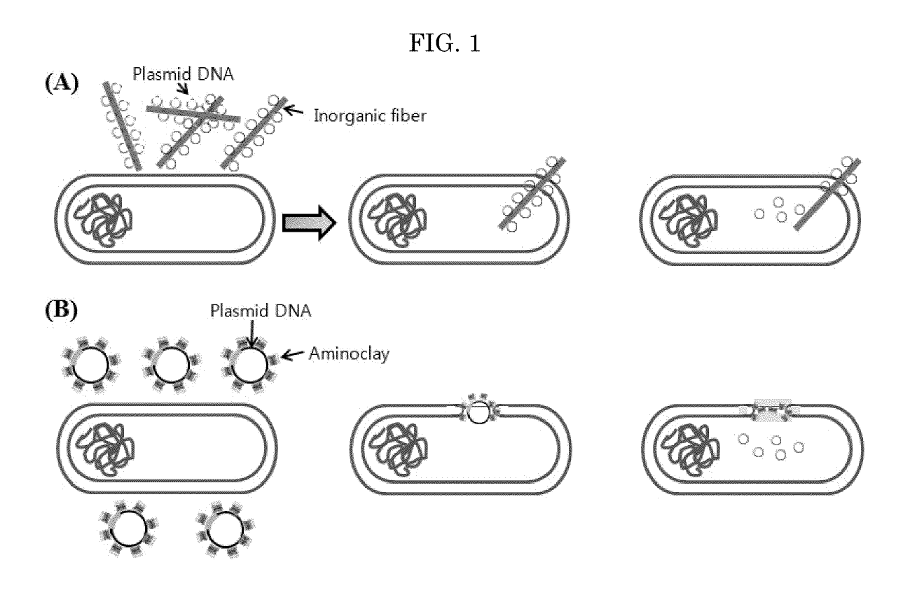

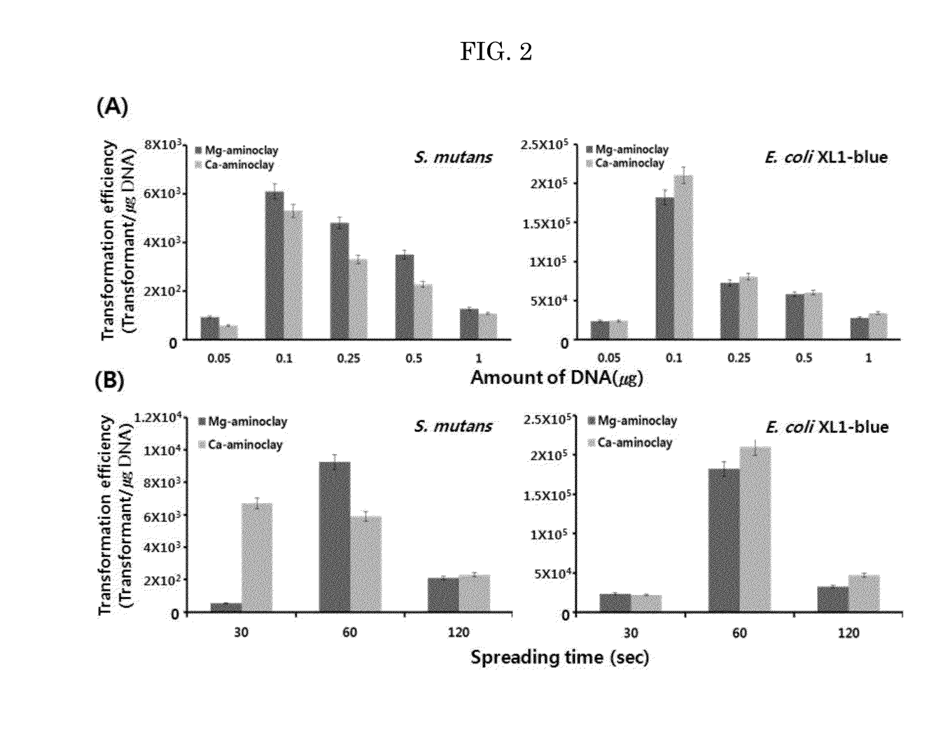

By using the prepared competent cells, the DNA-coated aminoclay was introduced to cells by spreading-induced frictional forces (FIG. 1B) based on the working mechanism of bacteria transformation as caused by Yoshida effect (FIG. 1A). As a result of test with various concentrations of aminoclay-DNA complex, the optimum conditions for transformation was found to be 0.25 mg when the cells of 4 to 6×10⁸ CFU were used as a host. The amount of the DNA and the time for spreading cells using the aminoclay/DNA which exhibit the optimum transformation efficiency under the aforementioned conditions were determined to be 0.1 μg and 1 minute, respectively, for both E. coli XL1-Blue and Streptococcus mutans. At that time, the bacteria transformation efficiency was about 2×10⁵ to about 6×10³ CFU/μg plasmid DNA (FIG. 2). In E. coli XL1-Blue, the transformation efficiency using calcium-aminoclay is higher than the case of using magnesium-aminoclay, in particular. However, an opposite result was obtained from Streptococcus mutans. The transformation efficiency decreases as the DNA loading amount increases or the spreading time is extended. It is believed to be a phenomenon resulting from, with an increased DNA loading amount, a defective membrane or cell wall is induced by accumulation of the DNA-aminoclay complex in a cell membrane in an amount larger than the amount suitable for cellular internalization (i.e., ratio of incorporation to the inside). As such, it was confirmed again that adverse effects are caused by damages of a cell or inhibited fluidity due to deformed membrane structure. It was also found by viable cell counting test that partial cell death is caused in bacterial cells by shear stress when the spreading time is extended to induce frictional forces.

Meanwhile, when the plasmid DNA transformation was conducted using sepioloite as a control agent according to the method based on Yoshida effect (sepiolite: needle-like nano structure for DNA adsorption, Wilharm et al., 2010. J. Microbiol. Methods 80, 215-216), not even single colony was detected by using Streptococcus mutans as a host under the same conditions. As an additional control group, from the results obtained from Streptococcus mutans which has been harvested from various time points during entire growth period and tested according to a general transformation protocol (e.g., heat shock treatment and electroporation), no transformant was produced, either. Thus, it was proven that the transformation of wild type Streptococcus mutans can be relatively easily and effectively performed by the method of the present invention. In case of the transformation of wild type E. coli, the transformation via heat shock treatment using pBBR122 has transformation rate was shown to be the same or slightly lower (˜2 fold) than that of the treatment of the same cells with the aminoclay/DNA method.

### EXAMPLE 3

### Transformation Efficiency of Aminoclay/DNA Method for Industrial Microorganisms

Insertion of plasmid DNA using electroporation is performed for many bacterial strains and animal cells. Compared to a heat shock treatment, it has higher efficiency and broad host range of use, and thus allows transformation of fungi and animal cells. Thus, many industrial microorganisms not allowing transformation by a heat shock treatment can be transformed by an electroporation method, and the transformation efficiency of representative bacteria is as follows; Staphylococcus aureus: 1×10⁴ CFU/μg plasmid DNA, Streptococcus pyogenes: 1×10⁴ CFU/μg plasmid DNA, Corynebacterium glutamicum: 3×10⁵ CFU/μg plasmid DNA, Propionibacteria acnes: 1.5×10⁴ CFU/μg plasmid DNA, Bacillus subtilis: 1×10³ CFU/μg plasmid DNA, Pseudomonas aeruginosa: 3×10³ CFU/μg plasmid DNA, Lactobacillus plantarum: 2×10⁴ CFU/μg plasmid DNA, and Agrobacterium tumefaciens: 2×10⁵ CFU/μg plasmid DNA. Thus, although being affected by size of the plasmid DNA for use and the thickness of a cell wall, it is generally in the range of 1×10³ to 1×10⁶ CFU/μg plasmid DNA. When the aminoclay/DNA is applied to the same bacterial strain, the transformant can be found at the range from 1×10³ CFU/μg plasmid DNA (minimum) to 1×10⁴ CFU/μg (maximum) in case of Gram-positive bacteria. Further, in case of Gram-negative bacteria, when the aminoclay/DNA is applied to the same bacterial strain, the transformant can be found at the range from 1×10⁴ CFU/μg plasmid DNA (minimum) to 1×10⁶ CFU/μg (maximum) (Table 1). The experimental conditions at that time were as described in Examples 1 and 2. The optimum cell concentration corresponded to optical density of 0.7 to 1.3 as an absorbance at 600 nm, the use amount of aminoclay was 0.2 to 0.3 mg, and the concentration of the recombinant DNA was 0.1 to 0.2 μg. All the strains which have been used were a type strain. Taken together, from all cases, the transformant was observed at a similar level to a typical electroporation method. However, considering the amount of the DNA and number of the cells that are actually used for the transformation, it is a value indicating higher efficiency with less amount. Further, not requiring any expensive device for electroporation, it is also very industrially advantageous.

### EXAMPLE 4

### Transformation Efficiency of Aminoclay/DNA Method for Industrial Yeast and Lower Fungi

Examples of the representative lower fungi that are used industrially include Saccharomyces cerevisiae, Schizosaccharomyces pombe, Pichia pastoris, Dictyostelium discodeum, Candida albicans, and Aspergillus oryzae. Unlike bacteria, transformation of lower fungi requires delivery of a plasmid to a nuclear membrane in a cell. For such reasons, the heat shock treatment used for bacteria is not applicable, and thus an electroporation method and a commercially available kit for transformation are commonly used. Although it may be greatly affected by the size of a plasmid DNA, the transformation efficiency by those methods are generally in the range of 1×10³ to 1×10⁴ CFU/μg plasmid DNA. When the aminoclay/DNA is applied to the same bacterial strain, it was able to confirm that the transformant is produced at the range from 1×10³ CFU/μg plasmid DNA (minimum) to 1×10⁴ CFU/μg (maximum) (Table 1). The experimental conditions at that time were as described in Examples 1 and 2. The optimum cell concentration corresponded to optical density of 0.5 to 1.1 as an absorbance at 600 nm. In case of lower fungi which forms hyphae or pseudo hyphae or has an extreme case of morphogenesis, the cells were used in an amount of 0.02 to 0.07 mg in terms of dry cell weight. The amount of the aminoclay used for transformation was 0.4 to 0.8 mg and the concentration of the recombinant DNA was 0.5 to 1.1 μg. All the strains which have been used were a type strain. Taken together, from all cases that are tested in the example, the transformant rate was the same or higher than that of a known electroporation method or a commercially available kit. However, considering the amount of the DNA and number of the cells that are actually used for the transformation, it is a value indicating higher efficiency with less amount. Further, not requiring any expensive device for electroporation, a commercially available kit, or any special pre-treatment process, it is also believed to be a very industrially favorable result.

TABLE 1
Transformation efficiency of various transformation methods

Unit: CFU/μg Plasmid DNA

| | | Transformation method | | | |
|---|---|---|---|---|---|
| | | | | | Aminoclay/ |
| Strain name | | Heat-Shock | electroporation | Kit method | DNA |
| Bacteria | Staphylococcus aureus | — | 1 × 10⁴ | — | 1 × 10⁴ |
| | Streptococcus pyogenes | 1 × 10² | 1 × 10⁴ | — | 1 × 10⁴ |
| | Corynebacterium glutamicum | 1 × 10² | 3 × 10⁵ | — | 1 × 10⁴ |
| | Propionibacteria acnes | — | 1.5 × 10⁴ | — | 1 × 10⁴ |
| | Bacillus subtilis | 1 × 10³ | 1 × 10³ | — | 1 × 10⁴ |
| | Pseudomonas aeruginosa | — | 3 × 10³ | — | 1 × 10⁶ |
| | Lactobacillus plantarum | — | 2 × 10⁴ | — | 1 × 10⁶ |
| | Agrobacterium tumefaciens | 1 × 10⁴ | 2 × 10⁵ | — | 1 × 10⁶ |
| Yeast/ | Saccharomyces cerevisiae | — | 4.5 × 10³ | >1 × 10³ | 1 × 10³ |
| Fungi | Schizosaccharomyces pombe | — | 1.6 × 10³ | >1 × 10³ | 1 × 10³ |
| | Pichia pastoris | — | 2 × 10⁴ | >1 × 10³ | 1 × 10⁴ |
| | Dictyostelium discodeum | — | 1 × 10⁴ | >1 × 10³ | 1 × 10³ |
| | Candida albicans | — | 4 × 10³ | >1 × 10³ | 1.3 × 10⁴ |
| | Aspergillus oryzae | — | 1 × 10⁴ | >1 × 10³ | 1 × 10³ |

### EXAMPLE 5

### Stability of Transformed Plasmid and Cell Growth

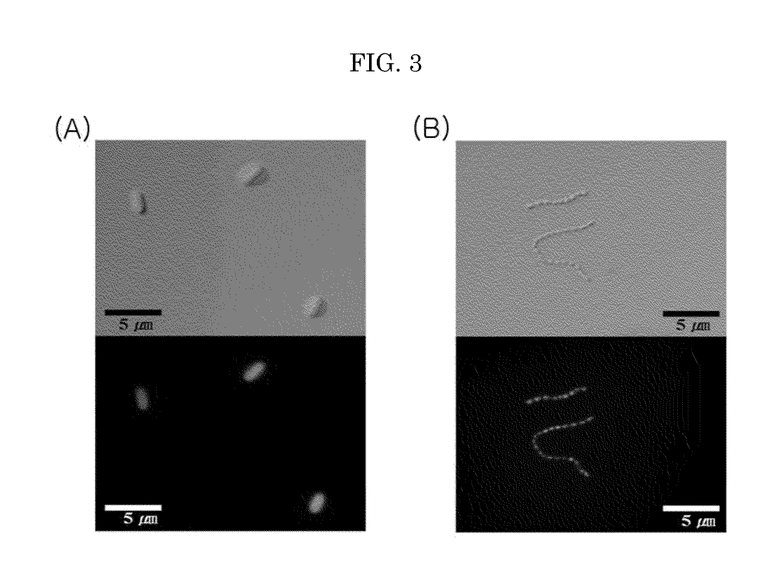

Each plasmid transformed in E. coli and Streptococcus mutans was stably maintained in the cells during the growth (i.e., until 80^{th} generation) without requiring any selection factor (i.e., antibiotics). When the growth is compared between the group of un-treated cells and the group of aminoclay-treated cells, no significant growth inhibition was shown. Further, when the cultured cells were analyzed by sodium dodecyl sulfate-polyacrylamide gel electrophoresis, a band corresponding to the stress-responding protein was not observed (data not shown). Because pBBR122 plasmid has a replication origin and an expression system functioning in a broad range of Gram-negative host, it was difficult to ensure an expression that is suitable for monitoring the expression of DNA inserted to Gram-positive cells. However, from both kinds of the recombinant microorganisms which have been transformed with pBBR122-gfp according to the aforementioned experiments, characteristic green fluorescence was shown under the culture conditions described above (FIG. 3). These results are the evidence supporting that the transformation system described in the present invention is operated well in a non-invasive manner, thus well express the insert gene encoding green fluorescence protein.

1. A method for transformation of a prokaryote or an eukaryote wherein the method includes mixing aminoclay, foreign nucleic acids, and a prokaryote or an eukaryote.

2. The transformation method according to claim 1, characterized in that the prokaryote is bacteria.

3. The transformation method according to claim 1, characterized in that the eukaryote is lower fungus or yeast.

4. The transformation method according to claim 1, characterized in that the aminoclay is hydrated.

5. The transformation method according to claim 1, characterized in that the mixing is performed by vortexing.

6. The transformation method according to claim 5, characterized in that the vortexing is performed for 30 seconds to 120 seconds.

7. The method according to claim 1, characterized in that culturing the mixture is further comprised after the mixing.

8. The transformation method according to claim 7, characterized in that culturing the mixture includes spreading the mixture on a plate medium by using frictional forces.

9. The transformation method according to claim 7, characterized in that selecting transformed prokaryote or eukaryote is further comprised after culturing the mixture.

10. A composition for transformation of a prokaryote or an eukaryote containing aminoclay.

11. The composition according to claim 11, characterized in that the prokaryote is bacteria.

12. The composition according to claim 11, characterized in that the eukaryote is lower fungus or yeast.

13. A kit for transformation of a prokaryote or an eukaryote containing aminoclay.


SEQUENCE LISTING (2 sequences)

SEQ ID NO: 1 - DNA, 18 bp, Artificial Sequence
  Primer
aaagtactatgagtaaag

SEQ ID NO: 2 - DNA, 20 bp, Artificial Sequence
  Primer
aaccgaattcttatttgtag